(19) United States
(12) Patent Application Publication
(10) Pub. No.: US 2026/0284644 A1
(43) Pub. Date: Sep. 24, 2026

(54) SULPHUR TOLERANT NOBLE METAL CATALYST FOR AROMATIC HYDROGENATION AND PRODUCING DEAROMATIZED SOLVENT

(71) Applicant: Hindustan Petroleum Corporation Limited, Karnataka (IN)

(72) Inventors: Kotni SANTHOSH, Karnataka (IN); Sunkara SUSHMA, Karnataka (IN); Ragireddy SUDARSHANREDDY, Karnataka (IN); Kanuparthy Naga RAJA, Karnataka (IN); Valavarasu GNANASEKARAN, Karnataka (IN); Sriram SUNDARARAMAN, Karnataka (IN)

(21) Appl. No.: 19/570,204

(22) Filed: Mar. 18, 2026

(30) Foreign Application Priority Data
Mar. 21, 2025 (IN) 202541025853

(51) Int. Cl. B01J 23/89; B01J 35/61; B01J 35/63; B01J 35/64; C07C 5/10

(52) U.S. Cl. CPC B01J 23/8926; B01J 35/615; B01J 35/635; B01J 35/647; C07C 5/10; C07C 2523/44

(57) ABSTRACT

The present invention provides a sulphur tolerant noble metal catalyst for hydrogenation. Particularly, the present invention provides a sulphur tolerant noble metal catalyst comprising of active metals such as palladium and platinum with promoter copper, impregnated on a silica/alumina support. The catalyst has a surface area in a range of 360 to 380 m²/g, pore volume in a range of 0.5 to 0.8 cc/g and pore size in a range of 6 to 8 nm. The invention further provides a method for catalytic hydrogenation of aromatics of hydrocarbon feedstock to obtain dearomatized solvents.

### FIELD OF THE INVENTION

The present invention relates to the field of catalysis. Particularly, the present invention provides a sulphur tolerant noble metal catalyst for hydrogenation. In particular, the present invention provides a catalyst of active metals such as palladium and platinum with promoter copper metal, impregnated on a silica/alumina support. The invention further provides a method for catalytic hydrogenation of aromatics of hydrocarbon feedstock.

### BACKGROUND OF THE INVENTION

Catalytic hydrogenation of aromatic compounds has attracted tremendous attention owing to the far-ranging industrial processes. The produced high value compounds are used in practical applications as important basic functional materials and organic synthesis intermediates. Many organic compounds and their derivatives are widely used in related fields such as household chemicals, pesticides, herbicides, medicines and fuels. On the other hand, with the increasing demand for diesel fuels, it is necessary and practically significant to eliminate toxic aromatic compounds. As the toxic aromatic compounds are a major source of air pollution. In fact, catalytic hydrogenation has been proved to be a feasible way to convert aromatics to saturated compounds. In view of the above, it is important to develop a highly efficient aromatic hydrogenation catalyst.

The noble metal catalysts have been used in the dearomatization of distillate feedstocks due to their higher hydrogenation activity as compared to the activity of non-noble metal (e.g. nickel, cobalt, molybdenum, and tungsten) catalysts. The high hydrogenation activity of noble metal catalysts reduces the required reaction temperatures for dearomatization and, thus, a greater temperature operating range for dearomatization processing. One difficulty, however, associated with the use of noble metal catalysts for the dearomatization of distillate feedstocks is associated with their sulfur sensitivity and tendency of being poisoned by the presence of sulfur that is contained in the feedstock being processed. One problem with the processing of certain refinery middle distillate streams is that they often contain a significant concentration of sulfur that makes the use of noble metal catalysts unsuitable for treating such refinery middle distillate. These streams have to first undergo a desulfurization treatment to reduce the sulfur content. Some of the prior art discussing the aromatic hydrogenation catalysts have been listed below.

US20100200462A1 discloses a aromatics hydrogenation catalyst composition which comprises a noble metal component and a support comprising zirconia, silica, and, optionally, alumina. The catalyst composition is manufactured by co-mulling silica, a zirconium compound, and, optionally, alumina to form a mixture that is formed into a shape, such as by extrusion to form an extrudate, with the shape being calcined and noble metal being incorporated into the shape. The catalyst composition may be used in the saturation of aromatic compounds.

MY155550 discloses an MCM-41 catalyst having a crystalline framework containing SiO₂ and a group IV metal oxide, such as TiO₂ or ZrO₂ is provided. The catalyst is low in acidity and is suitable for use in processes involving aromatic saturation of hydrocarbon feedstock.

US20160167017A1 discloses a method for preparing a sulfur-resistant catalyst for aromatics saturated hydrogenation, comprising the steps of: preparing noble metal impregnation solutions from a noble metal and deionized water or an acid solution; impregnating a carrier with the impregnation solutions sequentially from high to low concentrations by incipient impregnation; homogenizing, drying, and calcinating to obtain the sulfur-resistant catalyst for aromatics saturated hydrogenation. The catalyst for aromatics saturated hydrogenation prepared by the method according to the present invention is primarily used in processing low-sulfur and high-aromatics light distillate, middle distillate, atmospheric gas oil, and vacuum gas oil.

Most of the sulphur tolerant/resistant noble metal catalysts as available in the prior art work for hydrogenation of aromatic feed at pressure of around 55 bars and require large quantities of hydrogen making it less cost effective. Also, noble metal catalysts for aromatic hydrogenation reported in the prior art are shown to work at high temperatures above 200° C. This becomes highly energy intensive and also the hydrogenation being exothermic in nature, if the catalyst is active at low temperatures, the aromatization or the hydrogenation reaction is benefited in terms of achieving higher feed conversions and longer catalyst lifetime. So, there is a need to develop noble metal catalyst with increased hydrogenation activity, that can work at low pressure and temperature thus, making the hydrogenation of aromatic feed more cost effective in terms of using lesser quantity of hydrogen.

### OBJECTIVES OF THE INVENTION

It is an objective of the present invention to provide a sulphur tolerant noble metal catalyst for hydrogenation.

It is another objective of the present invention to provide a method for hydrogenation of aromatics of hydrocarbon feedstock.

It is another objective of the present invention to provide a method for hydrogenation of aromatics of hydrocarbon feedstock that has sulphur content.

### SUMMARY OF THE INVENTION

The present invention provides a sulphur tolerant noble metal catalyst for aromatic hydrogenation and producing dearomatized solvent, the catalyst comprises 0.05 to 0.3 wt. % of active metals, the active metals are palladium, platinum or their combination. The catalyst also comprises 0.08 to 0.2 wt. % of a promoter and 99.0 to 99.9 wt. % of support.

The active metals are selected form the combination of palladium and platinum in an elemental ratio of 3:1.

The promoter is copper metal.

The support is selected from silica, alumina or a combination thereof.

The catalyst is tolerant to 1 to 2 ppm sulphur impurity.

The catalyst has a surface area in a range of 360 to 380 m²/g, the catalyst has pore volume in a range of 0.5 to 0.8 cc/g, and the catalyst has pore size in a range of 6 to 8 nm.

The dearomatized solvent is dearomatized kerosene.

The present invention also provides a method for hydrogenation of aromatics present in a hydrocarbon feedstock with sulphur present upto 2 ppm, the method comprises the steps of contacting the hydrocarbon feedstock with said sulfur tolerant noble metal catalyst under hydrogenation conditions of temperature in a range of 160 to 200° C., pressure in a range of 30 to 35 bar and liquid hourly space velocity in a range of 0.5 to 1.5 h⁻¹.

The hydrocarbon feedstock consists of aromatics in a range of 4000 to 10000 ppm, and the aromatics of hydrocarbon feedstock are mono-aromatics, di-aromatics, tri-aromatic or a combination thereof.

The aromatics of hydrocarbon feedstock decreases to 70 to 120 ppm after hydrogenation.

### BRIEF DESCRIPTION OF THE ACCOMPANYING DRAWINGS

FIG. 1 illustrates the interaction of copper promoted noble metal catalyst with aromatic molecules.

FIG. 2 illustrates the effect of promoter copper metal on the hydrogenation activity of the sulphur tolerant noble metal catalyst.

FIG. 3 illustrates the effect of temperature on the hydrogenation activity of the sulphur tolerant noble metal catalyst at constant pressure of 30 bars.

### DETAILED DESCRIPTION OF THE INVENTION

For convenience, before further description of the present disclosure, certain terms employed in the specification, and examples are delineated here. These definitions should be read in light of the remainder of the disclosure and understood as by a person of skill in the art.

The terms used herein have the meanings recognized and known to those of skill in the art, however, for convenience and completeness, particular terms and their meanings are set forth below. The articles “a”, “an” and “the” are used to refer to one or to more than one (i.e., to at least one) of the grammatical object of the article. The terms “comprise” and “comprising” are used in the inclusive, open sense, meaning that additional elements may be included. It is not intended to be construed as “consists of only”. The term “at least one” is used to mean one or more and thus includes individual components as well as mixtures/combinations. Throughout this specification, unless the context requires otherwise the word “comprise”, and variations such as “comprises” and “comprising”, will be understood to imply the inclusion of a stated element or step or group of element or steps but not the exclusion of any other element or step or group of element or steps. The term “including” is used to mean “including but not limited to”. “including” and “including but not limited to” are used interchangeably.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods and materials are now described.

The present disclosure is not to be limited in scope by the specific embodiments described herein, which are intended for the purposes of exemplification only. Functionally equivalent products, compositions, and methods are clearly within the scope of the disclosure, as described herein.

The catalyst is designed such that, the promoter imparts more positive charge on the overall catalyst surface. This becomes the driving force between the negatively charged aromatic molecule and the positively charged metal catalyst surface. This enables the catalyst to function at lower operating pressures of 30 bar, otherwise the reported patents elsewhere demand high pressure more than 30 bar.

In an aspect, the present invention provides a sulphur tolerant noble metal catalyst for aromatic hydrogenation, the catalyst comprises 0.05 to 0.3 wt. % of active metals, the active metals are palladium, platinum or their combination. The catalyst also comprises 0.08 to 0.2 wt. % of a promoter and 99 to 99.9 wt. % of support.

In an embodiment of the present invention, the combination of palladium and platinum is in an elemental ratio of 3:1.

In an embodiment of the present invention, the promoter is copper metal.

In an embodiment of the present invention, the support is selected from silica, alumina or a combination thereof.

In an embodiment of the present invention, the catalyst is tolerant to 1 to 2 ppm sulphur impurity.

In an embodiment of the present invention, the catalyst has a surface area in a range of 360 to 380 m²/g, the catalyst has pore volume in a range of 0.5 to 0.8 cc/g, and the catalyst has pore size in a range of 6 to 8 nm.

In an embodiment of the present invention, the catalyst has a surface area of 369 m²/g, the catalyst has pore volume of 0.69 cc/g, and the catalyst has pore size of 7.52 nm.

In an embodiment of the present invention, the dearomatized solvent is dearomatized kerosene.

In another aspect, the present invention provides a method for hydrogenation of aromatics present in a hydrocarbon feedstock, the method comprises the steps of contacting the hydrocarbon feedstock with said sulfur tolerant noble metal catalyst under hydrogenation conditions of temperature in a range of 160 to 200° C., pressure in a range of 30 to 35 bar and liquid hourly space velocity in a range of 0.5 to 1.5 h⁻¹.

In an embodiment of the present invention, the hydrocarbon feedstock consists of aromatics in a range of 4000 to 10000 ppm, and the aromatics of hydrocarbon feedstock are mono-aromatics, di-aromatics, tri-aromatic or a combination thereof.

In an embodiment of the present invention, the aromatics of hydrocarbon feedstock decreases to 70 to 120 ppm after hydrogenation.

In an embodiment of the present invention, the aromatics of hydrocarbon feedstock decreases to 100 ppm after hydrogenation.

### EXAMPLES

The present disclosure is further illustrated by reference to the following examples which are for illustrative purposes only and does not limit the scope of the disclosure in any way. These examples are not intended to be inclusive of all aspects of the subject matter disclosed herein, but rather to illustrate representative features, methods, compositions, and results. These examples are not intended to exclude equivalents and variations of the present disclosure, which are apparent to one skilled in art.

### Example 1

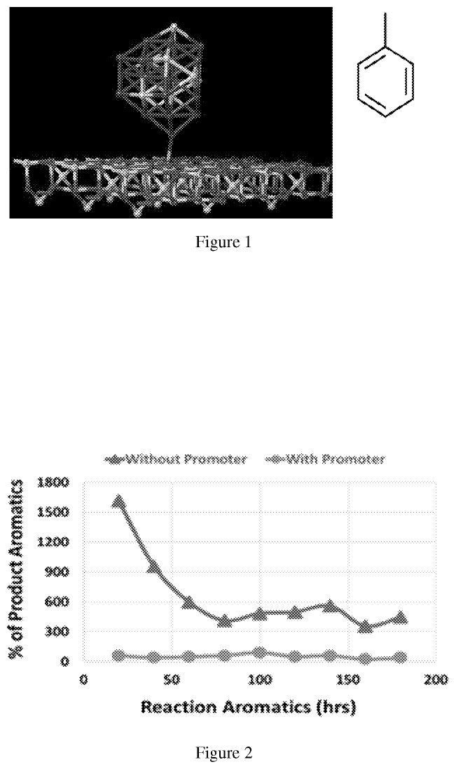

In this example, the interaction between the noble metal catalyst and the aromatics of the hydrocarbon feedstock was optimized by varying the charge on the catalyst with the introduction of various noble metals next to Palladium. It was observed that maximum positive charge was measured on the catalyst surface when copper is used as a promoter in the noble metal catalyst resulting in increased interaction between aromatics of hydrocarbon feed and the catalyst (FIG. 1) resulting in increased hydrogenation activity even at low pressures. The results are shown in table 1 below.

TABLE 1

| | | Charge on | Charge on | Overall |
|---|---|---|---|---|
| S. No. | Bimetallic | Palladium | Promoter | Charge |
| 1 | Pd | 0 | — | 0 |
| 2 | Pd₃Pt₁ | −0.0088 | +0.7564 | 0.747 |
| 3 | Pd₃Sn₁ | −7.29 | +7.73 | 0.44 |
| 4 | Pd₃Au₁ | −1.1484 | +1.4184 | 0.27 |
| 5 | Pd₃Ru₁ | −1.0201 | +1.3762 | 0.3561 |
| 6 | Pd₃Cu₁ | −2.1341 | +2.9876 | 0.8535 |

### Example 2

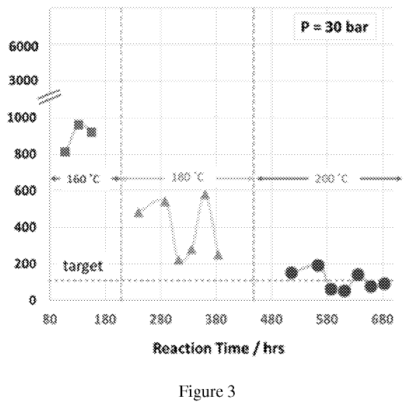

In this example lab scale development of the sulphur tolerant noble metal catalyst was performed. The feed used contained aromatics at 8000 ppm and sulphur content of 2 ppm and product aromatics concentrations was targeted at 100 ppm. The results showed that without promoter the product aromatics concentration decreased to 300 to 600 ppm at pressure of 30 bars, but it was reported that when pressure was increased to above 55 bars the aromatics concentration decreased to 100 ppm. With the promoter in the sulphur tolerant noble metal catalyst the product aromatics concentration decreased to less than 100 ppm even at 30 bars pressure (FIG. 2). The hydrogenation activity of sulphur tolerant noble metal catalyst with varying temperatures at a pressure of 30 bars is shown in FIG. 3.

We claim:

1. A sulphur tolerant noble metal catalyst for aromatic hydrogenation and producing dearomatized solvent, wherein the catalyst comprises:
0.05 to 0.3 wt. % of active metals, wherein the active metals are selected from palladium, platinum or a combination thereof;
0.08 to 0.2 wt. % of a promoter; and
99 to 99.9 wt. % of support.

2. The catalyst as claimed in claim 1, wherein the active metals are selected form the combination of palladium and platinum in an elemental ratio of 3:1.

3. The catalyst as claimed in claim 1, wherein the promoter is copper metal.

4. The catalyst as claimed in claim 1, wherein the support is selected from silica, alumina or a combination thereof.

5. The catalyst as claimed in claim 1, wherein the catalyst is tolerant to 1 to 2 ppm sulphur impurity.

6. The catalyst as claimed in claim 1, wherein the catalyst has a surface area in a range of 360 to 380 m²/g, wherein the catalyst has pore volume in a range of 0.5 to 0.8 cc/g, and wherein the the catalyst has pore size in a range of 6 to 8 nm.

7. The catalyst as claimed in claim 1, wherein the dearomatized solvent is dearomatized kerosene.

8. A method for hydrogenation of aromatics present in a hydrocarbon feedstock, wherein the method comprises:
contacting the hydrocarbon feedstock with the sulfur tolerant noble metal catalyst of claim 1 under hydrogenation conditions of temperature in a range of 160 to 200° C., pressure in a range of 30 to 35 bar and liquid hourly space velocity in a range of 0.5 to 1.5 h⁻¹.

9. The method as claimed in claim 8, wherein the hydrocarbon feedstock consists of aromatics in a range of 4000 to 10000 ppm, and wherein the aromatics of hydrocarbon feedstock are mono-aromatics, di-aromatics, tri-aromatic or a combination thereof.

10. The method as claimed in claim 8, wherein the aromatics of hydrocarbon feedstock decreases to 70 to 120 ppm after hydrogenation.
